(19) United States
(12) Patent Application Publication
(10) Pub. No.: US 2006/0149292 A1
(43) Pub. Date: Jul. 6, 2006

(54) Device for facilitating the insertion of a guidewire into the tip of intervascular guidewire guided device

(76) Inventors: Merril Knudtson, Calgary (CA); Spencer F. Smith, Dugald (CA); Ronald Michaluk, Birdshill (CA); Carey Ian Macrae, Winnipeg (CA)

Correspondence Address: LERNER GREENBERG STEMER LLP, P O BOX 2480, HOLLYWOOD, FL, 33022-2480, US

(21) Appl. No.: 11/027,347

(22) Filed: Dec. 30, 2004

(51) Int. Cl. A61F 11/00

(52) U.S. Cl. 606/108

(57) ABSTRACT

The invention provides a device for threading an end of a guidewire into a catheter, the catheter having a tip connected to an inner lumen of the catheter, the inner lumen dimensioned to receive the guidewire therethrough. The device comprises: a guidewire receiving port; a catheter tip receiving port; a channel connecting the guidewire receiving port to the catheter tip receiving port. The channel is dimensioned to pass the outer thickness of the guidewire therethrough and the channel is dimensioned to block passage of the outer thickness of the catheter therethrough. A contiguous section of the guidewire receiving port, the catheter tip receiving port and the channel form an open passageway from which the guidewire and catheter, once the guidewire is threaded into the catheter, are disengaged from the device. The open passageway may be covered by a flap mounted to the device which releasably cover,s the contiguous open section of the guidewire receiving port, the catheter tip receiving port and the channel.

### FIELD OF THE INVENTION

This invention relates to a device facilitating the insertion of a guidewire into the lumen of a catheter through the tip of the catheter. Catheters that are threaded in this manner are commonly called over the wire catheters or monorail catheters and are presently used in cardiology, radiology, and neurology. They are used in cardiology in procedures such as percutaneous transluminal coronary angioplasty (PTCA).

### BACKGROUND

PTCA is a technique used to dilate an area of arterial blockage with the help of a catheter that has an inflatable balloon at its tip.

In classical PTCA procedures, a guiding catheter is percutaneously introduced into the cardiovascular system of a patient and advanced therein until the distal tip of the guiding catheter is disposed within the aorta adjacent the ostium of the desired coronary artery. A guidewire is an extremely thin wire with a flexible tip. Typically the physician or operator inserts a guidewire through the guiding catheter into the coronary arteries of the heart. The tip of the guidewire is then guided across the blockage and advanced beyond it. The physician controls the movement and direction of the guidewire by gently manipulating the end that sits outside of the patient.

This guidewire can now serve as a “guide” over which a catheter may be threaded. The catheter will have a hollow lumen extending from the end that first enters the patient through all or a portion of the length of the catheter. To insert the catheter into a patient, the proximal tip of the guidewire (the end outside the patent) is fed into the lumen of the catheter through the distal Lip of the catheter. The distal tip of the catheter is then fed along the guidewire, into the patient's body and through the cardiovascular system until the tip of the catheter is positioned at a target site in the coronary arteries. The end of the catheter may be variously modified to include a balloon to expand arterial narrowings, to deploy a cylindrical device known as a stent to prevent arterial wall recoil after balloon expansion, or to do other intraarterial manipulations.

As will be appreciated by those skilled in the art, threading the one end of the guidewire into the lumen of the catheter through the catheter tip can be physically challenging because of the miniscule dimensions of the parts involved. Typically the guidewire bas a diameter of 0.004″ and the catheter tip has an inner diameter of approximately 0.016″-0.017″. The current approach relies on the technical ability of the physician to insert or thread the guidewire into the catheter at its tip.

Threading the guidewire into the lumen of the catheter through a small opening at the tip of the catheter can be difficult and time consuming, creating a potentially dangerous delay when quick action is required to safeguard the patient.

Additional damage can be encountered during manual insertion of the guidewire into the catheter tip, including kinking, crimping, or otherwise injuring the catheter tip, balloon or stent.

Some of the latest generations of stents have a drug coating on their surface. Since the stent is close to the tip of the catheter, typically, the physician must tip the catheter tip to assist in guiding the guidewire into the tip, thereby touching the stent. However, touching the stent may alter the effectiveness of the drug because some of the drug may be displaced.

Accordingly, what is needed is a hands-off approach, permitting the operator to rapidly and safely thread the guidewire into the catheter lumen without touching the stent located at the tip of the catheter. The present invention provides a device that (i) relieves the technical skill factor required to effectively thread the guidewire into the catheter lumen and (ii) provides a barrier to minimize hand contact with the stent when the catheter is being threaded onto the guidewire.

### SUMMARY OF THE INVENTION

In accordance with one aspect of the present invention, there is provided a device for threading an end of a guidewire into a catheter, the catheter having a tip corrected to an inner lumen of the catheter, the inner lumen dimensioned to receive the guidewire therethrough, wherein each of the guidewire and the catheter has an outer thickness, the device comprising: a guidewire receiving port; a catheter tip receiving port; and a channel connecting the guidewire receiving port to the catheter tip receiving port; wherein the channel is dimensioned to pass the outer thickness of the guidewire therethrough and the channel is dimensioned to block passage of the outer thickness of the catheter therethrough, and wherein a contiguous section of the guidewire receiving port, the catheter tip receiving port and the channel form an open passageway from which the guidewire and catheter, once the guidewire is threaded into the catheter, are disengaged from the device.

In accordance with another aspect of the present invention, there is provided, a device as described herein, further comprising a flap mounted to the device which releasably covers the contiguous open section of the guidewire receiving port, the catheter tip receiving port and the channel.

In accordance with another aspect of the present invention, there is provided a device as described herein wherein the flap is hingedly affixed to the device.

In accordance with yet a further aspect of the present invention, there is provided a device as described herein wherein the flap is deformable and releasably covers the contiguous open section of the guidewire receiving port, the catheter receiving port and the channel, through pressure applied externally to the flap.

In accordance with yet another aspect of the present invention, there is provided a device as described herein wherein the flap is fully releasable from the device.

In accordance with still another aspect of the present invention, there is provided a device as described herein wherein the fully releasable flap is mounted to the device by a heat seal or by adhesive.

In accordance with another aspect of the present invention, there is provided a device as described herein wherein the flap is tearable.

In accordance with yet a further aspect of the present invention, there is provided the device as described herein wherein the guidewire receiving port and the catheter tip receiving port are each funnel shaped with each funnel narrowing towards the channel.

In accordance with another of the present invention, there is provided the device as described herein wherein the channel is dimensioned where the channel meets the catheter tip receiving port to hold the outer thickness of the catheter at the tip of the catheter in a snug fit.

Additional aspects of the invention will become apparent to those skilled in the art upon reference to the detailed description taken in conjunction with the provided figures.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements shown. In the drawings, the same reference numerals are employed for designating the same elements throughout the several figures. In the drawings:

FIG. 1 is a top plan view of one embodiment of the invention without a flap or from which the releasable flap has been removed;

FIG. 2A is a top plan view of tie embodiment of FIG. 1 with one embodiment of the releasable flap shown in place;

FIG. 2B is a top plan view of a variation of the embodiment shown in FIGS. 1: and 2A, wherein the releasable flap is not fully releasable;

FIG. 3 is a perspective view of the embodiment shown in FIG. 1;

FIG. 4 is bottom plan view of the embodiment shown in. FIG. 1;

FIG. 5 is a front elevational view of the embodiment shown in FIG. 1;

FIG. 6 is a rear elevational view of the embodiment shown in FIG. 1; and

FIG. 7 is a top plan view of the embodiment shown in FIG. 1 illustrating a guidewire being thread into a catheter.

### DETAILED DESCRIPTION OF THE PREFERRED EMBODIMENTS

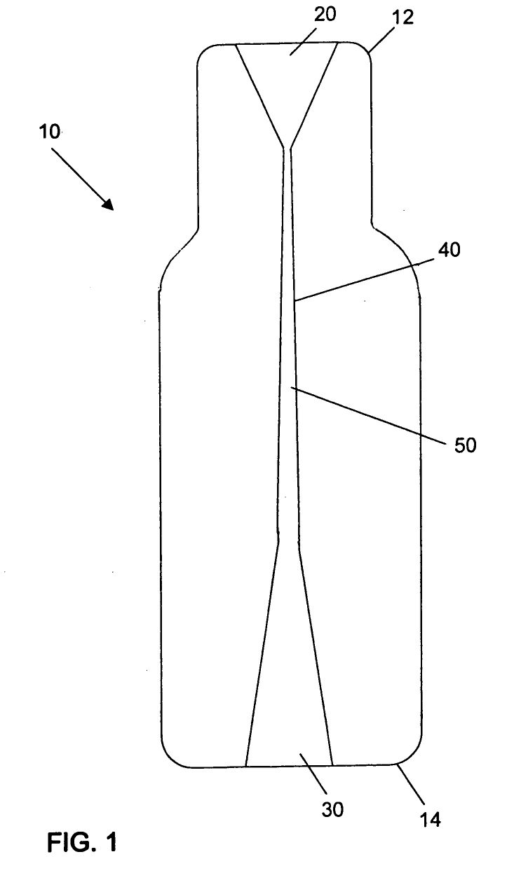

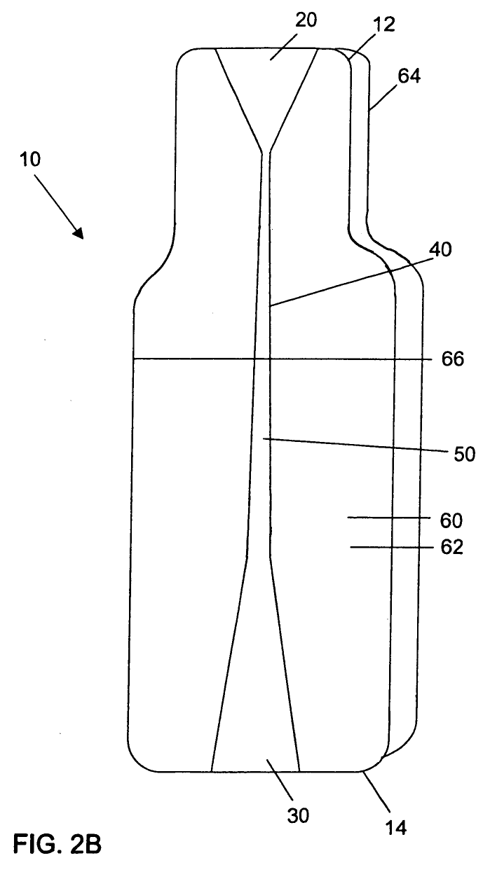

FIG. 1 shows an embodiment of device 10, as viewed from the top. In this embodiment, device 10 has the basic form of a rectangular block with a guidewire receiving port 20 at one end thereof 12 and a catheter receiving port 30 at the opposite end thereof 14 with a channel. 40 joining guidewire receiving port 20 to catheter receiving port 30. As can best be seen in the perspective view of FIG. 3, each of guidewire receiving port 20, catheter receiving port 30 amid channel 40 is a recessed void space at the top surface 16 of device 10, opposite from bottom surface 18 of device 10. The three of guidewire receiving port 20, catheter receiving port 30, and channel 40 together form a passageway 50 from one end 12 of device 10 to the other 14.

Device 10 may take any shape that allows for a passageway to be placed therein joining one end 12 to the other 14. In the embodiment of FIG. 1, device 10 is more narrow at end 12 than at end 14. The shape could be described as “bottle-shaped” or “violin shaped”. This shape merely allows a person using device 10 to more easily tell end 12 from end 14. In the embodiment of FIG. 1, guidewire receiving port 20 is found at end 12, the more narrow end of device 10. As a person using the device will know that a guidewire is more narrow than a catheter, it will help him or her remember that guidewire receiving port 20 is found at end 12, which is more narrow.

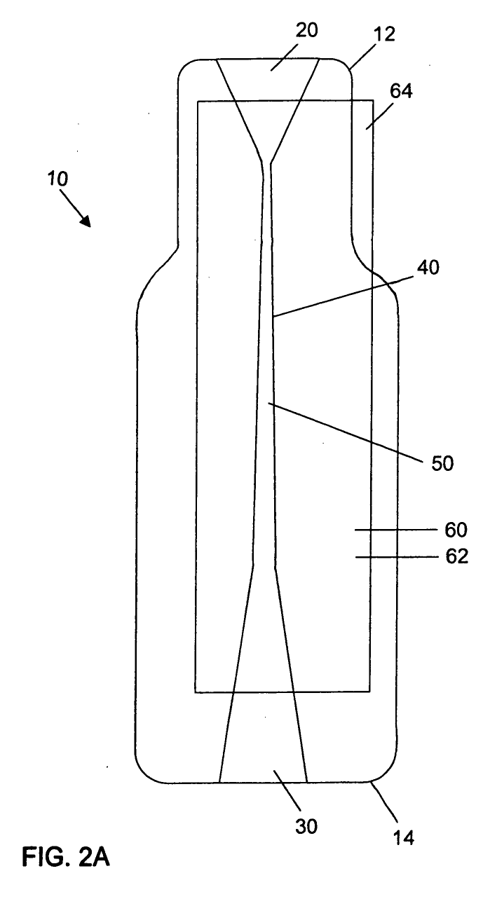

Passageway 50 may be covered by means of flap 60 (not shown in FIG. 1). Flap 60 can take any form so long as it releasably covers passageway 50. Flap 60 can be fully detachable from device 10 or partially detachable from device 10. In an embodiment where flap 60 is fully detachable, flap 60 cain take the form of a film or tape that is attached to top surface 16 of device 10 that can be peeled off of the top surface 16 of device 10. In such an embodiment, flap 60 can be attached to top surface 16 of device 10 by means of a heat seal or adhesive. FIG. 2A shows a top plan view of such an embodiment wherein flap 60 is in the form of a clear film 62, mounted on the top surface 16 of device 10. Clear film may be removed by pulling on tab 64. In the Embodiment shown in FIG. 2A, tab 64 is shown at end 12 of device 10, towards guidewire receiving port 20. However, tab 64 may be located anywhere on film 62 to allow access for the person using device 10 to grip tab 64 to remove film 62. In this embodiment, film 62 is fully removable from top surface 16 of device 10, but it need not be fully removable in a broader aspect of the invention. All that is required for this embodiment is that film 62 release from top surface 16 to such a degree to allow passageway 50 to be fully open along top surface 16. The importance of flap 60, it, this embodiment in the form of film 62, covering the open surface of passageway 50 comprising guidewire receiving port 20, catheter receiving port 30, and channel, 50, being releasable to an extent to allow passageway 50 to be open along top surface 16 will be seen below when use of the device to thread a guidewire into the lumen of a catheter is explained in detail. In the embodiment shown in FIG. 2B, film 62 is only partially removable from top surface 16 of device 10. Film 62 may be fixedly attached to top surface 16 at a seam 66 on the opposite side of tab 64. By being fixedly attached at seam 66, one can release film 62 by lifting tab 64 to separate film 62 from top surface 16 of device 10. Once lifted to expose passageway 50, film 62 stays attached to top surface 16 along seam 66. Because film 62 does not fully disengage from device 10, film 62 does not have to be disposed of as a separate piece of material, which is helpful in an operating room setting, where tidiness call be important.

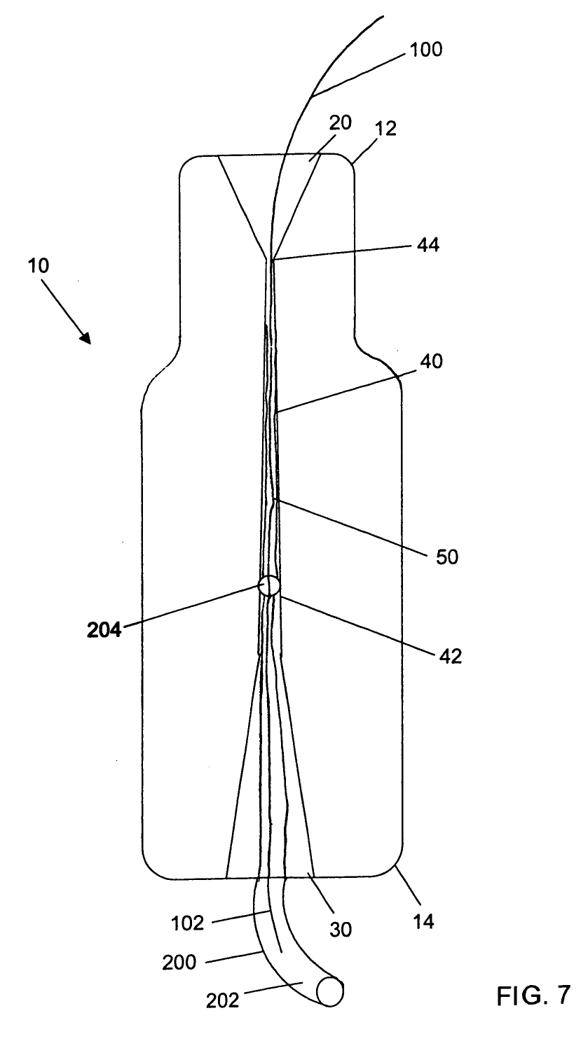

Use of device 10 to thread end 102 of guidewire 100 into lumen 202 of catheter 200 through tip 204 of catheter 200 will be described with the aid of FIG. 7. Tip 204 of catheter 200 is placed in catheter receiving port 30. In the embodiment shown in FIG. 7, catheter receiving port 30 is funnel-shaped, with the base of the triangular funnel being at end 14 of device 10, and the funnel narrowing as catheter receiving port 30 approaches channel 40. At the point 42, where catheter receiving port 30 meets channel 40, channel is preferably dimensioned to hold tip 204 of catheter 200 in a snug fit. The snug fit will allow catheter to be held in place in catheter receiving port 30 while end 102 of guidewire 100 is threaded into lumen 202 of catheter 200. However, the snug fit will still allow easy disengagement of catheter tip 204 from point 42 of channel 40 when catheter 200 is detached from device 10 once guidewire 100 is threaded into lumen 202 of the catheter. Once catheter tip 204 has been placed into catheter receiving port 30 and positioned adjacent to channel 40 at point 42, guidewire 100 may be threaded into the catheter. End 102 of guidewire 100 is fed into guidewire receiving port 20. In the embodiment shown in FIG. 7, guidewire receiving port 20 is funnel-shaped, with the base of the triangular funnel being at end 12 of device 10, and the funnel narrowing as guidewire receiving port 20 approaches channel. 40. Guidewire receiving port 20 narrows to point 44, where it meets channel 40. Channel 40 is dimensioned so that it has a diameter that is wider than that of guidewire 100, so as to allow guidewire to freely slide through channel 40. Guidewire end 102 is fed into guidewire receiving port 20, into channel 40, past point 44., towards point 42, where catheter tip 204 is being held. As guidewire end 102 is fed past point 42, it will be fed through catheter tip 204, into lumen 202 of catheter 200. In this embodiment guidewire receiving port 20 and catheter receiving port 30 are described as being in the shape of triangular funnels. However, any other shape that allows end 102 of guidewire 100 to be placed in guidewire receiving port 20 and tip 204 of catheter 200 to be placed in catheter receiving port 30, and end 102 and tip 204 to be fed towards channel 40, may be suitable.

Once guidewire end 102 is threaded into lumen 202 of catheter 200, device 10 may be disengaged from the threaded guidewire 100 catheter 200 combination by releasing flap 60. Flap 60 is not shown in FIGS. 1 and 7, but one can easily understand that if flap 60 took the form of film 62 illustrated in either of the embodiments of FIGS. 2A and 2B, all that one would have to do to release nap would be to pull back film 62 via tab 64. In the embodiment shown in FIG. 2A, film 62 is peeled back from top surface 16 of device 10, thereby exposing passageway 50. Device 10 can then be removed from the threaded guidewire 100 catheter 200 combination, thereby exposing catheter tip 204 which can be fed into an artery of a patent, for example, by further threading catheter 200 along guidewire 100. In the embodiment shown in FIG. 2B, film 62 is peeled back in a similar manner, exposing passageway 50. In this embodiment, however, film 62 does not filly disengage from device 10. Passageway 50 is still exposed, but film 62 still remains attached to device 10 along seam 66. Then device 10 can be disengaged as a unitary item with film 62, allowing for more easy disposal of device 10.

A person skilled in the art will realize that flap 60 can take other forms than the embodiments discussed above. All that is necessary is that the flap be able to releasably coffer a contiguous surface of passageway 50 that may be disengaged from the passageway, allowing for disengagement of the guidewire 100 catheter 200 combination once guidewire 100 has been threaded into lumen 202 of catheter 200. Flap 60 could take the form of a hinged cover that is hinged on one side of device 10 and is releasably attached to another side of device 10 on an opposite side of passageway 50. The releasable means of attachment could be by way of a snap fit of a groove on flap 60 and a tab on the “another” side of device 10, or other similar arrangement. In this embodiment, flap can be of unitary construction with remainder of device 10 such that all of device 10 is made of one material, and the hinge between flap 60 and the remainder of device 10 is a seam. This embodiment is possible when device 10 is made of a plastic material.

Device 10 docs not have to be fully disengageable from guidewire 100 catheter 200 combination. In this embodiment, flap 60 is deformable. Once guidewire 100 is threaded into lumen 202 of catheter 200, flap 60 may be deformed by bending or stretching to release the contiguous surface of flap 60 covering passageway 50. Alternatively, flap 60 may be arched, or otherwise loosely cover passageway 50 so that the operator may press down, for example with his or her thumb to cover passageway 50. In this embodiment, release of thumb pressure will cause flap 60 to separate from passage 50. Any of these embodiments of flap 60 will allow guidewire 100 catheter 200 combination to be released from the tight fit of passageway 50. Tip 204 of catheter 200 can be slid out from under flap 60 of device 10, thereby exposing tip 204 for insertion into the patient through the femoral artery. For example. The deformed, arched or lose embodiment of flap 60 has the effect of widening passageway 50, allowing device 10 to be disengaged from tip 204 of catheter 200, allowing device to be slid over catheter 200 to a site removed from tip 204, and the patient, as tip 204 is fed further into the patient.

In an alternative embodiment, flap 60 can take the form of a tearable film. Once guidewire 100 is fed into lumen 202 of catheter 200, the guidewire 100 catheter 200 combination may be simply pulled away from device 10, thereby ripping the tearable film and disengaging device 10.

When flap 60 takes the form of a film, the person skilled in the art would understand that the film may be attached to device 10 by means of adhesive, a heat seal or other suitable means. When flap 60 takes the form of a hinged cover, the person skilled in the art would understand that the non-hinged side of the cover may be attached to device 10 by means of adhesive, a releasable snap lock or other suitable means. When flap 60 takes the form of a deformable, arched or loose cover, the person skilled in the art would understand that such a cover may be attached to device by any suitable means on both sides of passageway 50 so long as the portion of the cover between the attachment means on both sides of passageway 50 is sufficiently deformable, arched or loose to permit guidewire 100 catheter 200 combination to disengage from the tight fit in passageway 50.

In a preferred embodiment, flap 60 will leave portions of one or both of guidewire receiving port 20 and catheter tip receiving port 30 uncovered a portion thereof close by ends 12 and 14 of device 10, respectively. These uncovered portions can assist an operator in placing catheter tip 204 in catheter tip receiving port 30 and guidewire 100 in guidewire receiving port 20, by effectively forming a guiding lip to assist in insertion.

In another embodiment, the flap does not have to be present as part of device 10. In this embodiment, a flap that is external to the device may be placed over open passageway 50. External flap can be any surface which can removably cover passageway 50. The external flap may even be the operator's thumb. However, when the operator uses his or her thumb, the thumb may come in contact with a stent which may be close to catheter tip 204. However, even when used with the operator's thumb as a flap, hand contact with the stent will be reduced from the alternative of not using device 10, since then the operator could likely handle the stent with both the thumb and the index finger when gripping catheter tip 204.

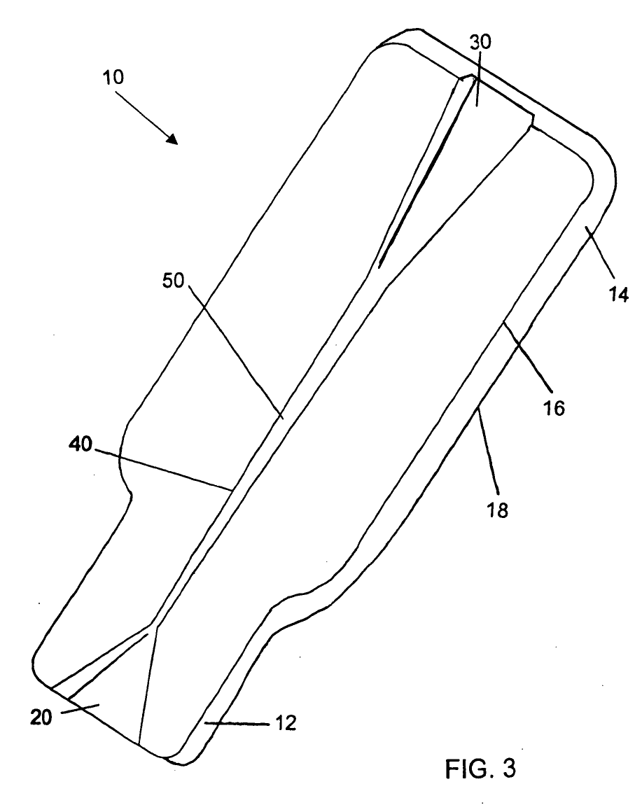

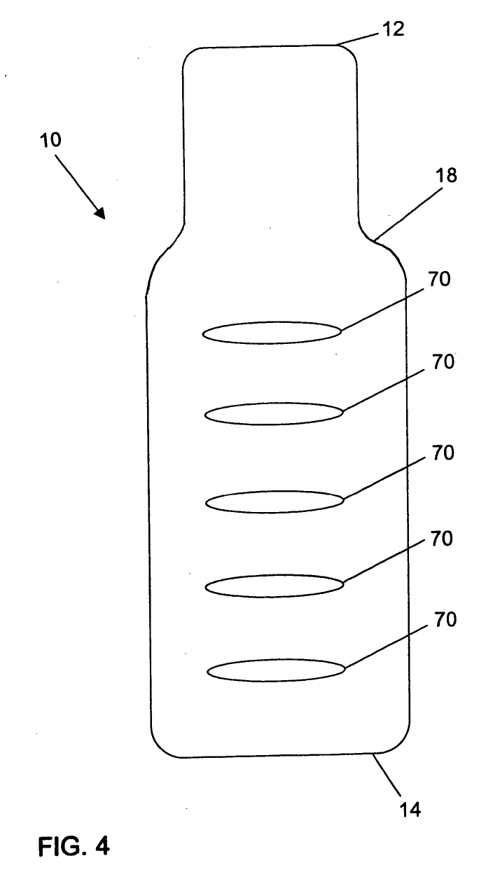

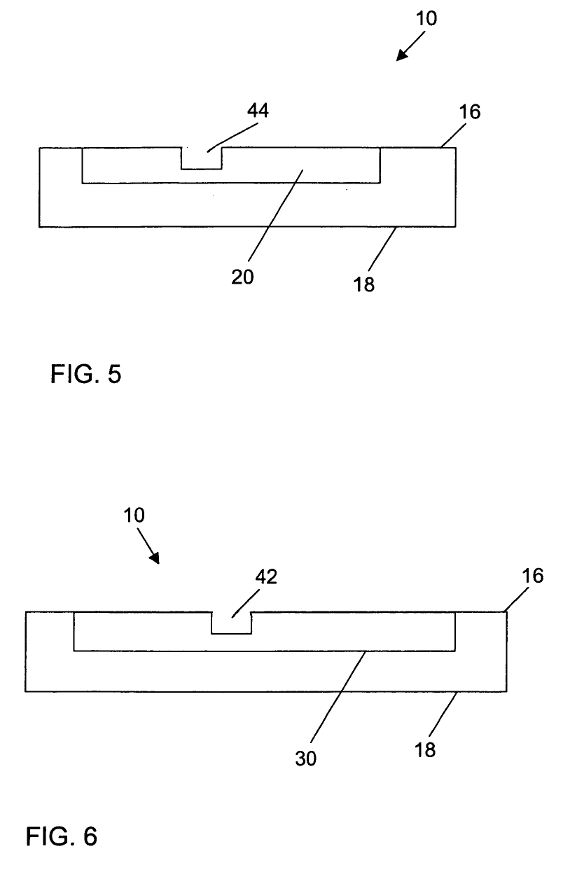

In the embodiment shown in the bottom plan view of FIG. 4, device 10 is fitted with a series of ridges 70. Having ridges 70 on the bottom surface 18 of device 10 allows one to pick up device 10 more easily once it has been placed on a surface, such as a table.

Device 10 is preferably made of sterilizable material. Device 10 may comprise sterilizable plastic, stainless steel, nitinol, latex, wood, or a combination thereof.

From the foregoing, it will be observed that numerous variations and modifications may be effected without departing from the true spirit and scope of the novel concept of this invention.

1. A device for threading an end of a guidewire into a catheter, the catheter having a tip connected to an inner lumen of the catheter, the inner lumen dimensioned to receive the guidewire therethrough, wherein each of the guidewire and the catheter as an outer thickness, the device comprising:
a guidewire receiving port;
a catheter tip receiving port; and
a channel connecting the guidewire receiving port to the catheter tip receiving port;
wherein the channel is dimensioned to pass tile outer thickness of the guidewire therethrough and the channel is dimensioned to block passage of the outer thickness of the catheter therethrough, and wherein a contiguous section of the guidewire receiving port, the catheter tip receiving port and the channel form an open passageway from which the guidewire and catheter, once the guidewire is threaded into the catheter, are disengaged from the device.

2. The device of claim 1, further comprising a flap mounted to the device which releasably covers the contiguous open section of the guidewire receiving port, the catheter tip receiving port and the channel.

3. The device of claim 2, wherein the flap is hingedly affixed to the device.

4. The device of claim 2, wherein the flap is deformable and releasably covers the contiguous open section of the guidewire receiving port, the catheter receiving post and the channel through pressure applied externally to the flap.

5. The device of claim 2, wherein the flap is fully releasable from the device.

6. The device of claim 5, wherein the fully releasable flap is mounted to the device by a heat seal.

7. The device of claim 5, wherein the fully releasable flap is mounted to the device by adhesive.

8. The device of claim 2, wherein the flap is tearable.

9. The device of claim 1, wherein the guidewire receiving port and the catheter tip receiving port are each funnel shaped, with each funnel narrowing towards the channel.

10. The device of claim 1, wherein said channel is dimensioned where the channel meets the catheter tip receiving port to hold the outer thickness of the catheter at the tip of the catheter in a snug fit.
